(19) United States
(12) Patent Application Publication
(10) Pub. No.: US 2022/0024872 A1
(43) Pub. Date: Jan. 27, 2022

(54) ISOLATION AND CHARACTERIZATION OF ANTICANCER COMPOUND FROM SESUVIUM PORTULACASTRUM (L.) L.

(71) Applicant: Sathvika Chintalapani, Telangana (IN)

(72) Inventors: Sathvika Chintalapani, Telangana (IN); Lakshmi Narasu Mangamoori, Hyderabad (IN)

(21) Appl. No.: 17/277,201

(22) PCT Filed: Feb. 13, 2020
(86) PCT No.: PCT/IN2020/050142

§ 371 Date: Mar. 17, 2021

(30) Foreign Application Priority Data
Feb. 25, 2019 (IN) 201941007336

(51) Int. Cl. C07D 213/16; A61K 36/185

(52) U.S. Cl. CPC C07D 213/16; A61K 36/185; A61K 2236/51; A61K 2236/15; A61K 2236/39; A61K 2236/17

(57) ABSTRACT

The invention discloses a novel anticancer compound, 3, 4, 5-trimethyl-2-(4-(8-methylnonyl)phenyl)pyridine extracted from Sesuvium portulacastrum (S. portulacastrum) having the structure as represented by Formula (I). The invention also discloses a method of extraction of this anticancer compound, 3, 4, 5-trimethyl-2-(4-(8-methylnonyl)phenyl)pyridine from S. portulacastrum. The invention further relates to characterization of the anticancer compound, 3, 4, 5-trimethyl-2-(4-(8-methylnonyl)phenyl)pyridine and study of its effect on the cancer cell cycle progression and expression of genes involved in apoptosis.

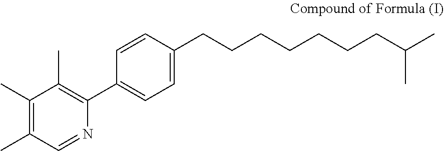 [SMILES: Cc1cnc(-c2ccc(CCCCCCCC(C)C)cc2)c(C)c1C]

### CROSS-REFERENCE TO RELATED PATENT APPLICATION(S)

This application is a National Stage application of International Patent Application No. PCT/IN2020/050142, filed on Feb. 13, 2020, which claim priority to Indian Provisional Patent Application No. 201941007336 filed on Feb. 25, 2019; the disclosures of which are incorporated herein by reference.

### TECHNICAL FIELD

The invention relates to a novel anticancer compound 3, 4, 5-trimethyl-2-(4-(8-methylnonyl)phenyl)pyridine extracted from Sesuvium portulacastrum (S. portulacastrum). The invention also relates to a method of extraction of this anticancer compound 3, 4, 5-trimethyl-2-(4-(8-methylnonyl)phenyl)pyridine from Sesuvium portulacastrum (S. portulacastrum). The invention further relates to characterization of the compound 3, 4, 5-trimethyl-2-(4-(8-methylnonyl)phenyl)pyridine and study of its effect on the cancer cell cycle progression and expression of genes involved in apoptosis.

### BACKGROUND

Chemotherapy, radiation, surgery and immunotherapy are often used individually or in combination to treat cancer. The chemotherapeutic agents are not only cytotoxic to tumor cells but also effect the proliferation of normal cell giving rise to side effects like nausea and vomiting, alopecia, and myelosuppression.

In some cases, the effectiveness of cancer drugs is limited by their insolubility and instability and low absorption in the body (Akindele et al., 2015). Further, the cancer cells often develop resistance to chemotherapeutic agents that are commonly used (Kakde et al., 2011).

In order to overcome these challenges, an alternative treatment, which is based on natural sources especially from the plant kingdom, is needed. Plants have a long history of use in the treatment of cancer. Also, the edible plants with anticancer properties if consumed regularly can prevent the occurrence of cancer. As crude extracts have less anticancer activity, purified anticancer compound with better activity was identified and isolated and characterized.

Sesuvium portulacastrum has been used in traditional medicine as a remedy for fever, kidney disorders and scurvy (Rojas et al., 1992). The plant is used on the Senegal coast as a haemostatic and a decoction of it is considered to be the best-known antidote for stings of venomous fish. (Lokhande et al., 2013). Environmentally the plant has an ability to survive under different abiotic stress conditions like salinity, drought, and heavy metal accumulation. (Slama et al., 2008).

The Journal of Ethnopharmacology (2006; 103(1): 85-89) discloses chemical composition and biological activities of essential oil from the leaves of Sesuvium portulacastrum. The essential oil exhibited antibacterial activity against Acetobacter calcoacetica, Bacillus subtillis, Clostridium sporogenes, Clostridium perfringens, Escherichia coli, Salmonella typhii, Staphylococcus aureus and Yersinia enterocolitica. The oil also exhibited antifungal activity against Candida albicans, Aspergillus niger, Aspergillus flavus and Penicillium notatum.

The anticancer compounds known in the art effects the proliferation of normal cells in addition to cancer cells and cause side effects like nausea and vomiting, alopecia and myelosuppression. Hence, there is need to develop anticancer compounds with better activity and fewer side effects.

Primary objective of the invention is to provide a novel anticancer compound with less side effects.

Another objective of the invention is to provide a novel anticancer compound obtained from natural source.

Another objective of the invention is to provide a novel anticancer compound 3, 4, 5-trimethyl-2-(4-(8-methylnonyl)phenyl)pyridine obtained from plant source.

Another objective of the invention is to provide a novel anticancer compound 3, 4, 5-trimethyl-2-(4-(8-methylnonyl)phenyl)pyridine obtained from Sesuvium portulacastrum (S. portulacastrum).

Another objective of the invention is to provide a method for extraction of anticancer compound 3, 4, 5-trimethyl-2-(4-(8-methylnonyl)phenyl)pyridine from Sesuvium portulacastrum (S. portulacastrum).

Another objective of the invention is to characterize the anticancer compound 3, 4, 5-trimethyl-2-(4-(8-methylnonyl)phenyl)pyridine and study its effect on the cancer cell cycle progression and expression of genes involved in apoptosis.

### SUMMARY

Accordingly, the present invention provides a novel anticancer compound isolated from Sesuvium portulacastrum

One of the aspects of the invention is to provide a compound of Formula (I)

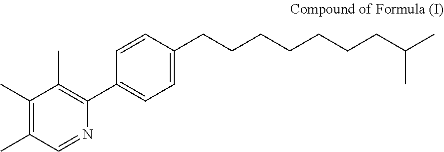 [SMILES: Cc1cnc(-c2ccc(CCCCCCCC(C)C)cc2)c(C)c1C]

or a pharmaceutically acceptable salt thereof.

In some embodiment, the compound of Formula (I) according to the invention is 3, 4, 5-trimethyl-2-(4-(8-methylnonyl)phenyl)pyridine. In some other embodiment, the compound of Formula (I) according to the invention is obtained by isolation from Sesuvium portulacastrum.

In some embodiment, the compound of Formula (I) is an anticancer agent. In some other embodiment, the compound of Formula (I) according to the invention is anticancer compound effective against breast cancer, human triple negative breast cancer MDA-MB-231 cells estrogen positive breast cancer MCF-7 cells, human neuroblastoma IMR-32 cells and human colon cancer HCT-116 cells.

In yet another aspect of the invention, there is provided a process for the isolation of compound of Formula (I) from Sesuvium portulacastrum,

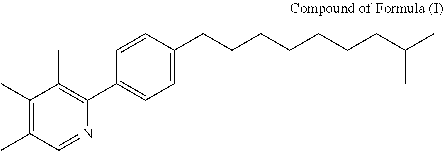 [SMILES: Cc1cnc(-c2ccc(CCCCCCCC(C)C)cc2)c(C)c1C]

said process comprising the steps of:
i. obtaining powder of dried whole plant of Sesuvium portulacastrum,
ii. extracting the powdered Sesuvium portulacastrum with one or more solvent,
iii. concentrating the extract obtained in step (ii) to obtain crude extract,
iv. optionally, fractionating the crude extract by column chromatography,
v. separating and purifying the crude extract obtained in step (iii) or the fractions obtained in step (iv) by preparative TLC and
vi. isolating the compound of Formula (I).

In some embodiment, the solvent used in step (ii) of the above described process for the isolation of compound of Formula (I) is selected from the group comprising of methanol, ethanol, acetone, hexane, ethyl acetate, dichloromethane, diethyl ether or mixture thereof. In preferred embodiment, the solvent used in step (ii) is diethyl ether.

In some embodiment, the column chromatography in the above described process for the isolation of compound of Formula (I) is carried by using the solvent system comprising hexane, ethyl acetate and dichloromethane in 50:5:45 ratio. In some other embodiment, the preparative TLC in the above described process for the isolation of compound of Formula (I) is carried by using the solvent system comprising hexane, ethyl acetate, dichloromethane and acetone in 49:5:44:2 ratio.

In another aspect of the invention, there is provided the use of compound of Formula (I) or a pharmaceutically acceptable salt thereof for the treatment of cancer.

In yet another aspect of the invention, there is provided a method for treating cancer in a subject comprising administering an effective amount of compound of Formula (I) or a pharmaceutically acceptable salt thereof.

In yet another aspect of the invention, there is provided a pharmaceutical composition comprising a pharmaceutically effective amount of the compound of Formula (I) and one or more pharmaceutical excipient.

With the foregoing and other advantages and features of the invention that will become hereafter apparent, the nature of the invention may be more clearly understood by reference to the following detailed description of the invention in conjunction of the accompanying drawings and the appended claims.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1. Anticancer activity of crude extracts (100-500 μg/ml). (a) effect of extracts on human breast cancer cells (MDA-MB-231); (b) effect of extracts on human breast cancer cells (MCF-7); (c) effect of extracts on human neuroblastoma cells (IMR-32); (d) effect of extracts on human colon cancer cells (HCT-116).

FIG. 2. Preparative TLC of fractions 1˜4 showing the separation of compounds 1-12. a—compound was mixed with fraction 4.

FIG. 3. Preparative TLC of fractions 5-8 showing the separation of compounds 13-27. Compounds 10 and 12 from fraction 5 are the same as compounds obtained from fraction 4.

FIG. 4. Identification of anticancer compound using on TLC of diethyl ether extract.

FIG. 5. Preparative TLC of crude diethyl ether extract.

FIG. 6. Anticancer activity of anticancer compound (40-200 μg/ml) at 24 h, 48 h and 72 h. (a) effect on human breast cancer cells (MDA-MB-231); (b) effect on human breast cancer cells (MCF-7); (c) effect on human neuroblastoma cells (IMR-32); (d) effect on human colorectal cancer cells (HCT-116).

FIG. 7. Detection of genes by PCR. Lane 1: MDA-MB-231 control cells; lane 2: MDA-MB-231 cells treated with anticancer compound (96 μg/ml) for 72 h; lane 3: IMR-32 control cells; lane 4: IMR-32 cells treated with anticancer compound (87 μg/ml) for 72 h; control cells are treated with 0.2% of ethanol-ethyl acetate mixture at 1:1 ratio.

FIG. 8. Relative gene expression of genes with respect to β-actin gene in MDA-MB-231 cells.

FIG. 9. Relative gene expression of genes with respect to β-actin gene in IMR-32 cells.

FIG. 10. LC-MS Chromatogram of a blank and anticancer compound with peak numbers.

FIG. 11. Spectrum showing common mass impurities in LC-MS.

FIG. 12. Mass Spectrum of anticancer compound.

FIG. 13. ¹H NMR spectrum of the anticancer compound.

FIG. 14. ¹³C NMR spectrum of the anticancer compound.

FIG. 15 (a). 2D NMR Spectrum of anticancer compound—Heteronuclear Multiple-Bond Correlation Spectroscopy (HMBC).

FIG. 15 (b). 2D NMR Spectrum of anticancer compound—Heteronuclear Single-Quantum Correlation Spectroscopy (HSQC).

FIG. 15 (c). 2D NMR Spectrum of anticancer compound—Nuclear Overhauser Effect Spectroscopy (NOESY).

FIG. 16. Selected HMBCs, ¹H and ¹³C chemical shifts of the anticancer compound.

FIG. 17. Structure of anticancer compound. Molecular formula C₂₄H₃₅N; molecular weight 337.54 Da.

### DETAILED DESCRIPTION

The present invention has been carried out with a view to identify, isolate, purify and characterize an anticancer compound with fewer side effects from Sesuvium. portulacastrum.

Accordingly, the present invention provides a novel anticancer compound isolated from Sesuvium portulacastrum

One of the aspects of the invention is to provide a compound of Formula (I)

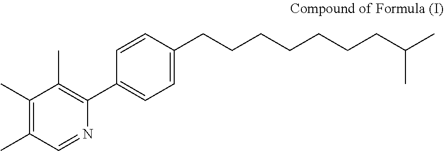 [SMILES: Cc1cnc(-c2ccc(CCCCCCCC(C)C)cc2)c(C)c1C]

or a pharmaceutically acceptable salt thereof.

In some embodiment, the compound of Formula (I) according to the invention is 3, 4, 5-trimethyl-2-(4-(8-methylnonyl)phenyl)pyridine.

In some embodiment, there is provided a compound of Formula (I) isolated from plant source. In some other embodiment, there is provided 3, 4, 5-trimethyl-2-(4-(8-methylnonyl)phenyl)pyridine of Formula (I) isolated from Sesuvium portulacastrum.

The term “pharmaceutically acceptable salt” as used herein refer to designate salts that are pharmaceutically acceptable, as defined herein, and which have the desired pharmacological activity of the parent compound. The term “pharmaceutically acceptable salt”, unless otherwise indicated, includes salts of acidic or basic groups that may be present in the parent compound.

The term “subject” as used herein refers to any animal, including mammals, such as mice, rats, other rodents, rabbits, dogs, cats, swine, cattle, sheep, horses, or primates, and humans.

The term “therapeutically effective amount” or “effective amount” as used herein refers to an amount of a compound, either alone or as a part of a pharmaceutical composition, that is capable of having any detectable, positive effect on any symptom, aspect, or characteristic of a disease or condition.

The terms “administration” or “administering” as used in herein refers to means of providing a compound of the present invention to a subject in a form that can be introduced into that subject's body in an amount effective for prophylaxis, treatment, or diagnosis, as applicable. Such forms may include e.g., oral dosage forms, injectable dosage forms, transdermal dosage forms, inhalation dosage forms, and rectal dosage forms.

The terms “treatment”, “treat” or “treating” as used herein refers a course of action (such as administering a compound or pharmaceutical composition) initiated after the onset of a symptom, aspect, or characteristics of a disease or condition so as to eliminate or reduce such symptom, aspect, or characteristics.

The term “pharmaceutical composition” means compositions in which the biological activity of the compound of Formula (I) has an effective effect, therefore, can be administered to a subject for therapeutic purposes. The term “pharmaceutical composition” also refers to a compound of Formula (I), optionally mixed with at least one pharmaceutically acceptable excipient such as, though not limited to carriers, stabilizers, diluents, dispersing agents, suspending agents, thickening agents, buffers or preservatives.

The term “pharmaceutically acceptable excipient”, as used herein refers to denote any material, which is inert in the sense that it substantially does not have any therapeutic and/or prophylactic effect per se. Such an excipient may be added with the purpose of making it possible to obtain the pharmaceutical composition, which have acceptable technical properties. The pharmaceutically acceptable excipient may include fillers, diluents, disintegrants, binders, lubricants, acidifying agents, alkalizing agents, preservatives, antioxidants, buffering agents, chelating agents, coloring agents, complexing agents, emulsifying and/or solubilizing agents, flavors and perfumes, humectants, sweetening agents, wetting agents, release controlling (e.g. release retarding or delaying polymers or waxes) agents, granulating agents, pigments, plasticizers, taste masking agents, tonicity adjusting agents and coating agents etc.

The term ‘stable composition’ as used herein refers that composition of the present invention does not undergo significant degradation under normal conditions of production or storage. In some embodiments, the term “stable”, referring to a composition or dosage form, means that the compound of Formula (I) and the pharmaceutical excipients remains unchanged in their chemical composition or physical state for a specified period of time and does not undergo significant destruction, aggregation or other changes.

In some embodiment, the present invention is also directed to pharmaceutically acceptable salts, stereoisomers, polymorphs, metabolites, analogues, and pro-drugs of the compound of Formula (I).

In some embodiment, the compound of Formula (I) according to the invention is an anticancer agent effective against various types of cancer. In some other embodiment, the compound of Formula (I) is effective against breast cancer and colon cancer. In some other embodiment, the compound of Formula (I) is an anticancer compound effective against human triple negative breast cancer MDA-MB-231 cells, estrogen positive breast cancer MCF-7 cells, human neuroblastoma IMR-32 cells and human colon cancer HCT-116 cells.

In yet another aspect of the invention, there is provided a process for the isolation of compound of Formula (I) from Sesuvium portulacastrum,

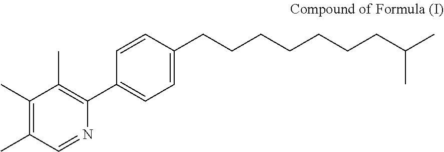 [SMILES: Cc1cnc(-c2ccc(CCCCCCCC(C)C)cc2)c(C)c1C]

said process comprising the steps of:
i. obtaining powder of dried whole plant of Sesuvium portulacastrum,
ii. extracting the powdered Sesuvium portulacastrum with one or more solvent,
iii. concentrating the extract obtained in step (ii) to obtain crude extract,
iv. optionally, fractionating the crude extract by column chromatography,
v. separating and purifying the crude extract obtained in step (iii) or the fractions obtained in step (iv) by preparative TLC and
vi. isolating the compound of Formula (I).

In some embodiment, the extraction in the above described process is carried by percolation or by Soxhlation method.

In some embodiment, the solvent used in step (ii) of the above described process for the isolation of compound of Formula (I) from Sesuvium portulacastrum is selected from the group comprising of methanol, ethanol, acetone, hexane, ethyl acetate, dichloromethane, diethyl ether or mixture thereof. In preferred embodiment, the solvent used in step (ii) is diethyl ether.

In some embodiment, the column chromatography in the above described process for the isolation of compound of Formula (I) is carried by using the solvent system comprising hexane, ethyl acetate and dichloromethane in 50:5:45 ratio. In some embodiment, the column chromatography is followed by preparative TLC for isolation of compound of Formula (I). In some other embodiment, the preparative TLC in the above described process for the isolation of compound of Formula (I) is carried by using the solvent system comprising hexane, ethyl acetate, dichloromethane and acetone in 49:5:44:2 ratio. In some embodiment, the preparative TLC is directly used for separating and purifying the crude extract obtained in step (iii) for isolation of the compound of Formula

In some embodiment, the crude extract is used for the treatment of cancer or the preparation of medicament/composition for the treatment of cancer.

In yet another aspect of the invention, there is provided a pharmaceutical composition comprising a pharmaceutically effective amount of the compound of Formula (I) and one or more pharmaceutical excipient. In some embodiment, the composition according to invention is a stable composition wherein the compound of Formula (I) retains its physical resistance or chemical resistance or both.

The pharmaceutical compositions according to invention can be formulated for oral administration in solid or liquid form, for parenteral or for vaginal, nasal, topical, or rectal administration. Pharmaceutical compositions of the present invention suitable for oral administration can be presented as discrete dosage forms, e.g., tablets, chewable tablets, caplets, capsules, powders, granules, liquids, and flavored syrups. Such dosage forms contain predetermined amounts of compound of invention, and may be prepared by methods of pharmacy well known to those skilled in the art. Such compositions and methods for their preparation can be found, for example, in ‘Remington's Pharmaceutical Sciences’, 19th Edition (Mack Publishing Company, 1995), the disclosure of which is incorporated herein by reference in its entirety.

The compound of the invention may also be administered in form of parenteral dosage form and suitable means for parenteral administration includes intravenous, intraarterial, intraperitoneal, intrathecal, intraventricular, intraurethral, intrasternal, intracranial, intramuscular and subcutaneous. Suitable devices for parenteral administration include needle (including micro needle) injectors, needle-free injectors and infusion techniques.

The compound of invention may be administered in form of parenteral dosage forms which are specifically sterile or capable of being sterilized prior to administration to a subject. Examples of parenteral dosage forms include solutions ready for injection, dry products ready to be dissolved or suspended in a pharmaceutically acceptable vehicle for injection, suspensions ready for injection, and emulsions. Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol) vegetable oils (such as olive oil) and injectable organic esters such as ethyl oleate, or suitable mixtures thereof. These compositions may also contain adjuvants such as preservative agents, wetting agents, emulsifying agents, and dispersing agents. It may also be desirable to include isotonic agents, for example, sugars, sodium chloride and the like or the preservatives for example, parabens, chlorobutanol, phenol, sorbic acid, and the like.

In some cases, in order to prolong the effect of a drug, it is often desirable to slow the absorption of the drug from injection, particularly from subcutaneous or intramuscular injection. The delayed absorption of a parenterally administered drug form is accomplished by dissolving or suspending the drug in an oil vehicle. In addition, prolonged absorption of the injectable composition may be brought about by the inclusion of agents which delay absorption such as gums, cellulose derivative, aluminum monostearate, gelatin etc.

The compound of invention may be formulated as suspensions, which in addition to the active compounds, may contain suspending agents, for example, sodium carboxymethylcellulose, methylcellulose, hydroxypropylmethylcellulose, sodium alginate, polyvinylpyrrolidone, tragacanth gum and acacia gum; dispersants or wetting agents are natural phosphatides, ethoxylated isostearyl alcohols, polyoxyethylene sorbitol and sorbitan esters, microcrystalline cellulose, aluminum metahydroxide, bentonite, agar-agar, tragacanth, and mixtures thereof. In some cases, and for more effective distribution, the compounds of the invention can be incorporated into slow-release or targeted-delivery systems such as polymer matrices, liposomes, and microspheres.

Injectable depot composition comprising compound according to invention provides a sustained release benefit agent delivery system implanted after injection into a patient's body. Injectable depot forms are made by forming microencapsulated matrices of the drug in bioerodible and biocompatible polymer such as glycolide, lactide, caprolactone or copolymers thereof, or are prepared by entrapping the drug in liposomes or microemulsions which are compatible with body tissues. Depending upon the ratio of drug to polymer and the nature of the particular polymer employed, the rate of drug release can be controlled. Examples of other biodegradable polymers include poly(orthoesters) and poly(anhydrides).

Parenteral compositions in form of sterile injectable aqueous or oleaginous suspensions may be formulated according to the known art using suitable dispersing or wetting agents and suspending agents. A sterile injectable solution, suspension or emulsion in a nontoxic, parenterally acceptable diluent or solvent are covered under the scope of the invention. Non-limiting examples of acceptable vehicles and solvents include water, Ringer's solution, isotonic sodium chloride solution, fixed oils, fatty acids and the like.

The injectable formulations can be sterilized by filtration through a bacterial-retaining filter or by incorporating sterilizing agents in the form of sterile solid compositions which can be dissolved or dispersed in sterile water or other sterile injectable medium just prior to use.

Solid dosage forms for oral administration may include capsules, tablets, pills, powders, and granules. In such solid dosage forms, compound of the invention is mixed with at least one inert pharmaceutically acceptable carrier such as citric acid, sodium citrate or dicalcium phosphate and/or a) fillers or extenders such as sugars or sugar alcohols including lactose, sucrose, glucose, dextrose, mannitol or sorbitol; b) binders such as tragacanth, starches, carboxymethylcellulose, alginates, gelatin, polyvinylpyrrolidinone, sugars, acacia, polyethylene glycol and the like; c) humectants such as glycerol; d) disintegrating agents such as hydroxyalkylcellulose, starches, agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as mineral or vegetable oil, glyceryl behenate, talc, alkaline stearates (calcium stearate, magnesium stearate), solid polyethylene glycols, sodium lauryl sulfate, and mixtures thereof. In the case of capsules, tablets and pills, the dosage form may also comprise buffering agents. In some cases, composition may be delayed release that release the compound of invention, in a certain part of the intestinal tract in a delayed manner. In some cases, the additional delay can be achieved with the help of retardant coatings such as water-insoluble waxes or polymers, for example acrylic resins, preferably poly (meth) acrylates, or water insoluble celluloses, preferably ethyl cellulose.

Compositions comprising compound of invention for rectal or vaginal administration in form of pessaries or suppositories can be prepared by mixing the compounds of this invention with suitable non-irritating carriers such as cocoa butter, polyethylene glycol or a suppository wax which are solid at ambient temperature but liquid at body temperature and therefore melt in the rectum or vaginal cavity and release the active compound.

Compositions for topical use and nasal administration may include ointments, creams, powders, sprays, patches, gels, liquid drops and inserts. Such compositions can be formulated in accordance with known methods. Compositions for administration by inhalation may take the form of inhalable powder, liquid or powder sprays and can be administrated in standard form using powder inhaler devices or aerosol dispensing devices. Powders and sprays can contain, in addition to the compounds of this invention, lactose, talc, silicic acid, aluminum hydroxide, calcium silicates and polyamide powder, or mixtures of these substances.

Liquid dosage forms for oral administration of compound of Formula (I) include pharmaceutically acceptable emulsions, microemulsions, solutions, suspensions, syrups and elixirs. The liquid dosage forms contain active ingredients and additionally contain inert diluents such as water or other solvents, solubilizing agents and one or more emulsifier selected from cationic emulsifiers, anionic emulsifiers or nonionic emulsifier. Non-limiting examples of emulsifier include lecithin, polysorbate 20, polysorbate 40, polysorbate 60, polysorbate 80, ethyl alcohol, isopropyl alcohol, ethyl carbonate, ethyl acetate, benzyl alcohol, benzyl benzoate, propylene glycol, 1,3-butylene glycol, dimethylformamide, polyoxyethylene (40) stearate, propylene glycol laurate, oils (in particular, cottonseed, groundnut, corn, germ, olive, castor, and sesame oils), glycerol, tetrahydrofurfuryl alcohol, polyethylene glycols or mixture thereof.

Besides compound of Formula (I), the oral compositions may include diluents and/or adjuvants such as wetting agents, emulsifying and suspending agents, sweetening, flavoring, and perfuming agents. Dosage forms for topical or transdermal administration of a compound of this invention include ointments, pastes, creams, lotions, gels, powders, solutions, sprays, inhalants or patches. The compound of the invention is admixed under sterile conditions with a pharmaceutically acceptable carrier and any needed preservatives or buffers as may be required. Ophthalmic formulation, ear drops, eye ointments, powders and solutions are also contemplated as being within the scope of this invention. The ointments, pastes, creams and gels may contain, in addition to an active compound of this invention, include animal and vegetable fats, oils, waxes, paraffins, starch, tragacanth, cellulose derivatives, polyethylene glycols, silicones, bentonites, silicic acid, talc and zinc oxide, or mixtures thereof.

Compounds of the invention may also be administered in the form of liposomes. Liposomal bilayer membranes of the liposomes are typically formed by lipids, i.e., synthetic or naturally-occurring amphiphilic molecules comprising spatially separated hydrophilic and hydrophobic domains. Liposomal bilayer membranes can also be formed by amphiphilic polymers and surfactants (polymersomes, niosomes). Any non-toxic, physiologically acceptable and metabolizable lipid capable of forming liposomes may be used, preferably the natural or synthetic phospholipids and phosphatidylcholines. The liposome according to invention may contain pharmaceutical excipients in addition to the compound of Formula (I) and be prepared according to methods known in art.

The amount of compound of invention in the pharmaceutical composition of this invention can be varied so as to obtain an amount of the active compound that is effective to achieve the desired therapeutic response for example severity of the condition being treated and the condition and prior medical history of the patient being treated; age, body weight, general health, sex and diet of the patient; type of compositions; mode of administration etc.

In another aspect of the invention, there is provided the use of compound of Formula (I) or a pharmaceutically acceptable salt thereof for the treatment of cancer. In some embodiment, the compound of Formula (I) or a pharmaceutically acceptable salt thereof is used in the preparation of medicament for the treatment of cancer. In some embodiment, the cancer is lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, or pituitary adenoma. In some other embodiment, the compound of Formula (I) or the composition/medicament thereof is used for the treatment of breast cancer or colon cancer.

In yet another aspect of the invention, there is provided a method for treating cancer in a subject comprising administering an effective amount of compound of Formula (I) or a pharmaceutically acceptable salt thereof. In some other embodiment, there is provided a method for treating breast cancer or colon cancer comprising administering an effective amount of compound of Formula (I) or a pharmaceutically acceptable salt thereof.

In some embodiment, the structure of compound of Formula (I) as 3, 4, 5-trimethyl-2-(4-(8-methylnonyl)phenyl)pyridine has been elucidated based on the data from LC-MS, ¹H NMR, ¹³C NMR, and 2D-NMR analysis.

Based on the structure of the anticancer compound it was concluded that anticancer compound is a pyridine alkaloid. Pyridine alkaloids are nitrogen-containing chemical compound found in plants. Naturally occurring pyridine alkaloids are known to have many biological and pharmacological properties (Jie & Gribble, 2000). Other Pyridine derived compounds that are synthesized chemically exhibited good anticancer activity against MCF-7 cells (El-Nagga et al., 2018). Pyridine derivative also showed a cell cycle arrest at the G₂/M phase in MCF-7 cells and liver cancer cells (HepG2 Cells) and induced apoptosis via p53 activation (Androutsopoulos and Spandidos, 2018).

The anticancer compound of Formula (I) that is isolated from diethyl ether extract of S. portulacastrum is novel and has not been isolated from any other plant species. The isolated compound exhibited good anticancer potential against MDA-MB-231, MCF-7, IMR-32 and HCT-116 cells. It exhibited very little effect on the normal cells showing that it can be developed into anticancer drug with fewer side effects. Morphological changes like cell shrinkage, reduction in cellular volume, increased number of floating or dead cells were observed in the cells treated with the isolated compound of Formula (I).

The compound of Formula (I) also inhibited cell cycle progression at G₂/M phase in MDA-MB-231 and at G₁ phase in IMR-32 Cells. Treatment of MDA-MB-231 cells with the compound of Formula (I) for 72 h, led to down regulation of Nrf2 and Bcl2 genes and up regulation of caspase-3 expression leading to apoptosis. Treatment of IMR-32 cells with the compound for 72 h resulted in apoptosis accompanied by up regulation of caspase-3 and downregulation of IKK-β and Nrf2 genes.

The anticancer compound that was isolated from S. portulacastrum showed good cytotoxic potential against cancer cells. Also, it exhibited a very little effect on the normal cells showing that it can be developed into an anticancer drug with fewer side effects.

The compound according to invention overcomes the resistance developed by cancer cells to chemotherapeutic agents that are commonly used.

Further, the structure can be used to chemically synthesize the anticancer compound and further analyze its potential as a therapeutic drug.

Also based on the structure of the compound, other lead anticancer compounds can be further developed.

### EXAMPLES

### Example 1: Isolation of Compound of Formula (I) from S. Portulacastrum

### Collection of Plant Material:

S. portulacastrum was collected from the forest area of Tirumala hills, Andhra Pradesh. The plant was cleaned thoroughly with water and shade dried in order to prevent thermal degradation. Air-dried plant was ground to powder and stored at a cool and dry place in dark airtight containers to avoid oxidation.

### Extraction of Crude Extracts:

For the extraction of crude bioactives, plant powder was initially diluted with different solvents with a solvent to sample ratio of 10:1 (v/w). The diluted plant sample was kept at room temperature undisturbed for 24 h. It was then kept in an orbital shaker for the next 48 h. Various solvents like ethanol, methanol, acetone, hexane and diethyl ether were used for the extraction process. The diluted sample was then filtered through Whatman No. 1 filter paper to obtain crude extracts. The extract was concentrated under reduced pressure at 40° C. using Rota evaporator. The dried crude concentrated extracts were weighed to calculate the yield percentage and stored at room temperature until further analysis.

### Effect of Crude Extracts on Cancer Cells:

Anticancer activity of crude extracts were evaluated against four cell lines, human triple negative breast cancer cells (MDA-MB-231), estrogen positive breast cancer cells (MCF-7), human neuroblastoma cells (IMR-32) and human colon cancer cells (HCT-116) that were procured from NCCS-Pune using 3-(4,5-Dimethylthiazol-2-Yl)-2,5-Diphenyltetrazolium Bromide (MTT) assay.

### MTT Cell Proliferation Assay:

In vitro cytotoxic activity of all the extracts was determined by MTT assay as described by Manikandan et al. (2017) with slight modification. After trypsinization, viable cells were counted by trypan blue exclusion in a hemocytometer and diluted with the medium.

The cells were seeded in 96 well plate at a density of 1-2×10⁴ cells per well. The plate was incubated at 37° C. in a 5% CO₂ atmosphere. The following day media was removed and the cells were treated with standard, doxorubicin (0.1-4 μg/ml) or plant extract at different concentrations (100-500 μg/ml). The extract was initially dissolved in 1:1 ratio of ethanol-ethyl acetate mixture and further diluted in medium to get the required concentrations. As plant extracts were dissolved in ethyl acetate and as ethyl acetate is not miscible with cell culture media, a mixture of ethanol-ethyl acetate in a ratio of 1:1 is used for all the cell culture experiments. The final mixture used for treating the cells contained not more than 0.5% of the ethanol-ethyl acetate mixture. Negative controls were treated with the same percentage of ethanol-ethyl acetate mixture.

The treated plate was incubated at 37° C. in a humidified incubator with 5% CO₂ for 24 h. After the incubation period, the media was removed, 100 μl of 0.1 mg/ml MTT reagent was added and incubated at 37° C. in a humidified incubator with 5% CO₂ for 1 h till purple MTT formazan crystals were visible under the microscope. The formazan crystals were dissolved by adding 100 μl DMSO and spectrophotometric readings were taken at 540 nm.

The cytotoxic activity of standard or plant extract was calculated as the percentage of cell growth inhibition by using the following formula.

$$1. Growth inhibition ( \% ) = \frac{( {A}_{Control} - {A}_{Sample} )}{{A}_{Control}} \times 100$$

Where A_{control} indicates the absorbance of control containing 0.5% ethanol-ethyl acetate mixture. Aₛₐₘₚₗₑ is the absorbance of treated cells at different concentrations. IC₅₀ values (the concentration of sample required for inhibition of 50% of cell growth) were obtained from the regression line.

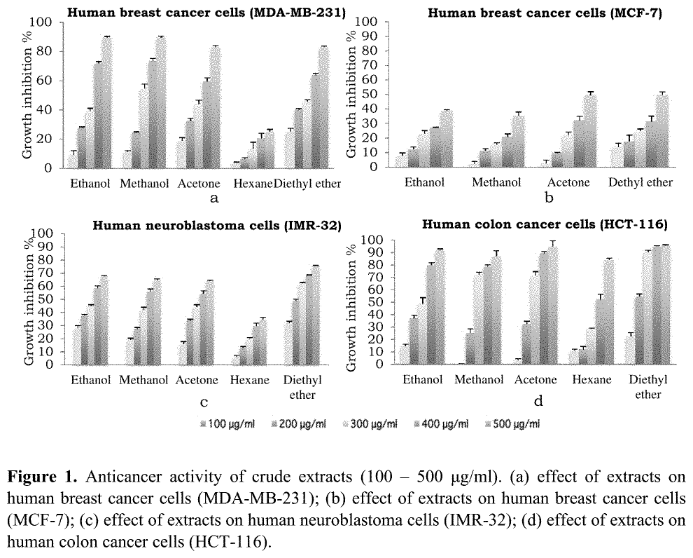

All the extracts showed a dose-dependent inhibition of cell proliferation in MDA-MB-231, MCF-7, IMR-32 and HCT-116 cells as shown in FIG. 1. Diethyl ether extract showed highest anticancer potential among all the crude extracts that is evident from its lowest IC₅₀ values shown in Table 1.

TABLE 1
IC₅₀ values of crude extracts and standard in MTT assay.

| | IC₅₀ (μg/ml) | | | |
|---|---|---|---|---|
| Extract/standard | MDA-MB-231 | MCF-7 | IMR-32 | HCT-116 |
| Ethanol | 312.49 ± 4.97^{aP} | 549.73 ± 14.67^{aq} | 326.34 ± 3.51^{abp} | 277.92 ± 6.82^{ap} |
| Methanol | 297.21 ± 2.02^{ap} | 741.46 ± 42.66^{bq} | 269.18 ± 8 45^{adp} | 248.86 ± 3.18^{acp} |
| Acetone | 315.65 ± 7.70^{ap} | 530 ± 19.11^{aq} | 364.53 ± 6.13^{bp} | 242.05 ± 7.01^{acr} |
| Hexane | 942.07 ± 74.82^{bp} | No activity | 703.40 ± 31.19^{cq} | 407.87 ± 0.40^{br} |
| Diethyl ether | 288.69 ± 6.53^{ap} | 521.50 ± 14.2^{aq} | 231.01 ± 6.32^{dpr} | 182.86 ± 4.29^{cr} |
| Doxorubicin | 0.83 ± 0.03^{cp} | 0.66 ± 0.04^{cp} | 0.50 ± 0.02^{ep} | 0.40 ± 0.02^{dp} |

Data are mean values of triplicates and expressed as mean ± SD. For each column, values with different letters (a-e) indicate significantly different (p ≤ 0.05). For each row, values with different letters (p-r) indicate significantly different (p ≤ 0.05).

### Fractionation of Crude Diethyl Ether Extract Using Column Chromatography and Preparative TLC:

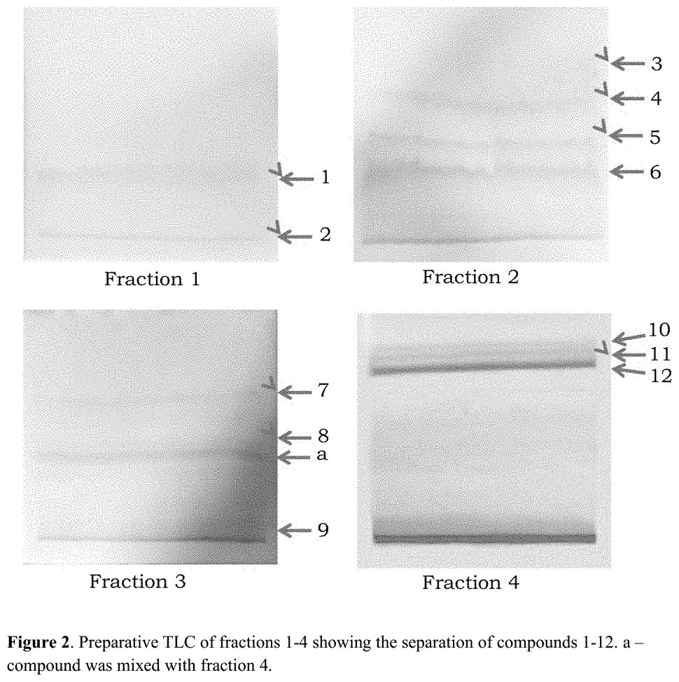

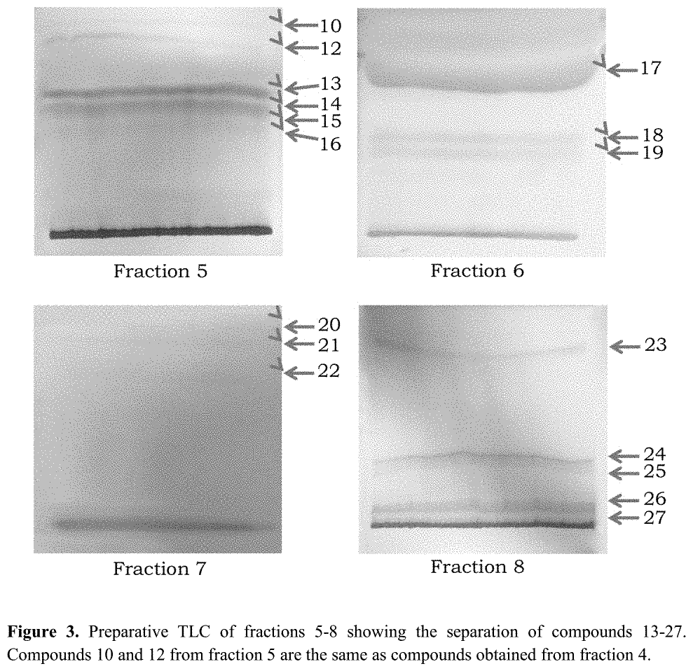

As diethyl ether extract showed the highest anticancer potential, it was fractionated using column chromatography. A total of 18 fractions were collected from column chromatography. The fractions, which gave the same TLC pattern, were combined to give fraction 1 to fraction 9. Solvent system was optimized for each fraction and each fraction was further purified using preparative TLC. The preparative TLC patterns of fractions 1-4 are shown in FIG. 2 and the respective solvent systems are shown in Table 2. FIG. 3 and Table 3 shows the preparative TLC patterns of fractions 5-8 and their solvent systems respectively.

TABLE 2
Solvent systems used for the separation of fractions 1-4

| Fraction No. | Solvent system |
|---|---|
| 1 | 100% Hexane |
| 2 | Ethyl acetate:hexane (5:95) |
| 3 | Ethyl acetate:hexane (7.5:92.5) |
| 4 | Ethyl acetate:hexane (12.5:87.5) |

TABLE 3
Solvent systems used for the separation of fractions 5-8

| Fraction No. | Solvent system |
|---|---|
| 5 | n-Hexane:ethyl acetate:dichloromethane (50:5:45) |
| 6 | n-Hexane:ethyl acetate:dichloromethane (45:15:40) |
| 7 | n-Hexane:ethyl acetate:dichloromethane (45:15:40) |
| 8 | n-Hexane:ethyl acetate:dichloromethane (45:15:40) |

A total of 27 compounds were isolated from eight fractions. Due to very small amounts being present in fraction 9, it was not separated using preparative TLC and was analyzed directly and termed as fraction 28. Further, the most polar band from all the fractions was combined and termed as fraction 29. All the compounds separated along with crude diethyl ether extract were analyzed for their anticancer potential against MDA-MB-231 cells at a concentration of 300 μg/ml using MTT assay as described above. Cytotoxic activity was expressed as percentage inhibition of cells at 300 μg/ml. Data is represented in Table 4.

TABLE 4
Anticancer activity of compounds/fractions separated. Data
are mean values of duplicates and expressed as mean ± SD.

| | Percentage |
|---|---|
| Compound/fractions | Inhibition |
| 1 | 49.44 ± 2.57 |
| 2 | 29.27 ± 0.99 |
| 3 | 17.65 ± 4.36 |
| 4 | 3.08 ± 1.19 |
| 5 | 21.71 ± 4.16 |
| 6 | 56.02 ± 1.58 |
| 7 | 52.52 ± 3.37 |
| 8 | 20.17 ± 1.58 |
| 9 | 71.71 ± 3.57 |
| 10 | 47.90 ± 3.96 |
| 11 | 80.53 ± 0.59 |
| 12 | 58.12 ± 2.57 |
| 13 | 66.39 ± 1.58 |
| 14 | 48.46 ± 1.98 |
| 15 | 53.50 ± 2.77 |
| 16 | 94.54 ± 0.59 |
| 17 | 68.07 ± 3.17 |
| 18 | 60.64 ± 4.95 |
| 19 | 81.79 ± 3.96 |
| 20 | 30.39 ± 4.16 |
| 21 | 30.11 ± 3.76 |
| 22 | 44.40 ± 0.20 |
| 23 | 74.09 ± 1.39 |
| 24 | 82.21 ± 0.99 |
| 25 | 85.99 ± 2.77 |
| 26 | 68.77 ± 3.76 |
| 27 | 66.67 ± 3.96 |
| 28 | 35.99 ± 2.18 |
| 29 | 74.93 ± 0.99 |
| Crude | 52.32 ± 3.15 |

Among all the compounds separated, compound 16 showed the highest anticancer activity that is evident from its highest percentage of inhibition. Also, the inhibition percentage of compound 16 (anticancer compound) is more than that of crude diethyl ether extract.

Separation and Purification Anticancer Compound Directly from the Crude Extract Using Preparative TLC:

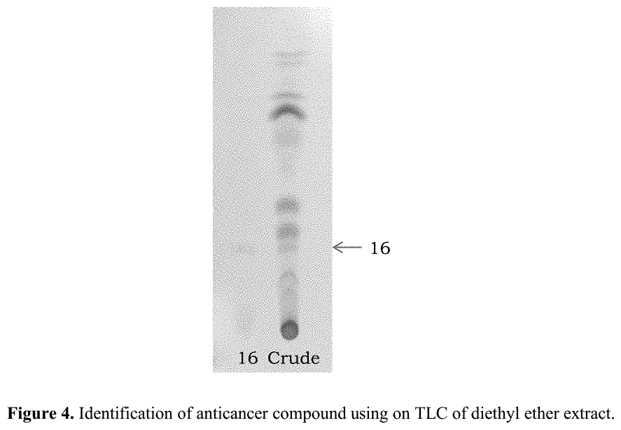

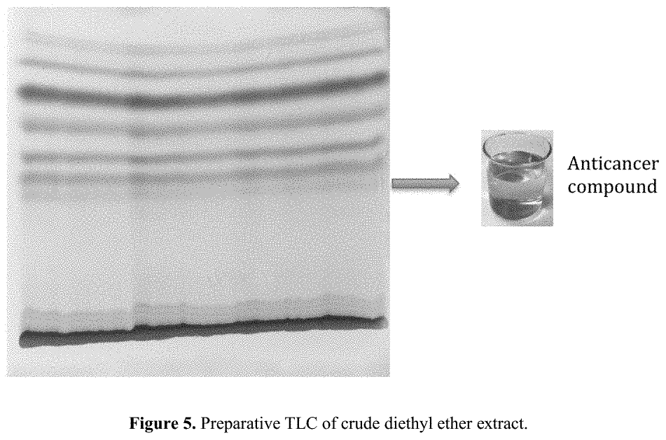

Initially, the compounds 16 (anticancer compound) was identified on the crude diethyl ether chromatogram developed in hexane:ethyl acetate:dichloromethane (50:5:45) as shown in FIG. 4. Later, the solvent system hexane:ethyl acetate:dichloromethane:acetone (49:5:44:2) was used to separate anticancer compound directly from crude diethyl ether extract using preparative TLC as shown in FIG. 5 (addition of 2% acetone solvent to the solvent system helped in the faster separation of compounds). The filtrate is collected and dried at 37° C. in hybridization chamber to obtain the anticancer compound in dried form. The anticancer compound was further purified.

### Example 2: Evaluation of the Anticancer Compound According to Invention

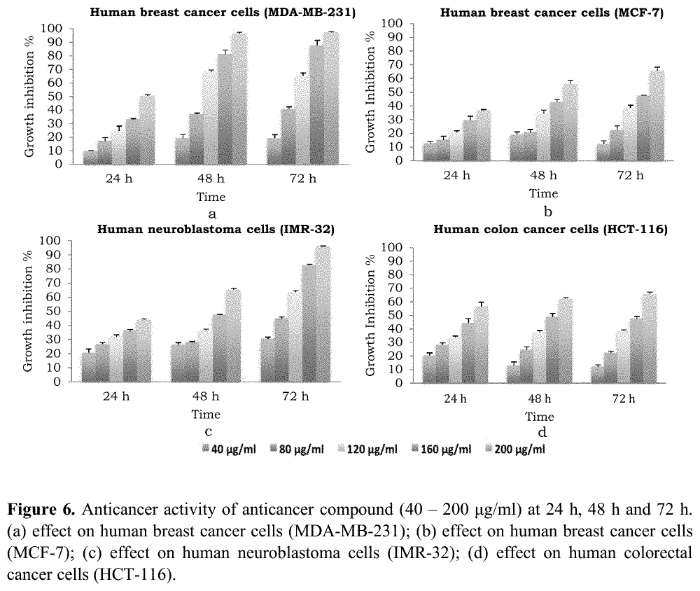

The anticancer potential of the anticancer compound against MDA-MB-231, MCF-7, IMR-32 and HCT-116 cells was evaluated at different concentrations (40, 80, 120, 160 and 200 μg/ml) and different incubation times (24 h, 48 h, 72 h) using MTT assay by the method described above. The results are shown in Table 5 and FIG. 6.

TABLE 5
IC₅₀ values of the anticancer compound in MTT assay.

| | IC₅₀ (μg/ml) | | | |
|---|---|---|---|---|
| Time (h) | MDA-MB-231 | MCF-7 | IMR-32 | HCT-116 |
| 24 | 199.25 ± 0.50^{ap} | 242.8 ± 5.52^{acp} | 223.92 ± 7.21^{ar} | 179.11 ± 6.12^{as} |
| 48 | 99.11 ± 1.60^{bp} | 183.25 ± 6.29^{bq} | 165.23 ± 0.87^{br} | 161.50 ± 5.02^{br} |
| 72 | 96.25 ± 1.75^{bp} | 154.52 ± 5.24^{cq} | 87.85 ± 0.21^{cp} | 158.65 ± 2.87^{bq} |

Data are mean values of triplicates and expressed as mean ± SD. For each column, values with different letters (a-c) indicate significantly different (p ≤ 0.05). For each row, values with different letters (p-s) indicate significantly different (p ≤ 0.05).

### Effect of the Anticancer Compound on Lymphocytes:

In order to know the side effects associated with the treatment of anticancer compound, the effect of the anticancer compound on the viability of human peripheral blood lymphocytes was determined using trypan blue exclusion assay. Along with the viability of lymphocytes, the viability of different cancer cells at that particular concentration and incubation time are shown in Table 6.

TABLE 6
Effect of anticancer compound on the viability of lymphocytes and cancer cells

| | | Viability percentage | | | | |
|---|---|---|---|---|---|---|
| Incubation | Concentration | | Human Cancer Cells | | | |
| Time (h) | (μg/ml) | Lymphocytes | MDA-MB-231 | IMR-32 | MCF-7 | HCT-116 |
| 24 | 400 | 90.40 ± 4.02 | 0 | 0 | 0 | 0 |
| 48 | 350 | 82.11 ± 1.42 | 0 | 0 | 0 | 0 |
| 72 | 300 | 74.94 ± 1.50 | 0 | 0 | 0 | 3.29 ± 3.15 |

Data are mean values of triplicates and expressed as mean ± SD

According to Food and Drug Administration, Center for Drug Evaluation and Research (CDER), it is considered as clinically significant, if there is more than 40% reduction in total lymphocytes (Hannet et al., 1992; Luster et al., 1993) and more than 75% reduction in total granulocytes (Johansen, 1983) after treatment with any therapeutic drug. At a concentration of 400 μg/ml, the viability of lymphocytes was found to be 91.04% when treated and incubated with anticancer compound for 24 h. Though higher concentrations of anticancer compound are required to inhibit the proliferation of cancer cells, the side effect caused by the anticancer compound is very limited even at that higher concentration.

In the case of doxorubicin (standard drug), the lymphocyte viability was reduced to 65% even at a concentration of 1 μg/ml when incubated for 24 h (Dash et al., 2013). The viability percentage of lymphocytes is still maintained at 74.94% when treated with anticancer compound at a concentration of 300 μg/ml for 72 h.

### Effect of the Anticancer Compound at Transcriptional Level:

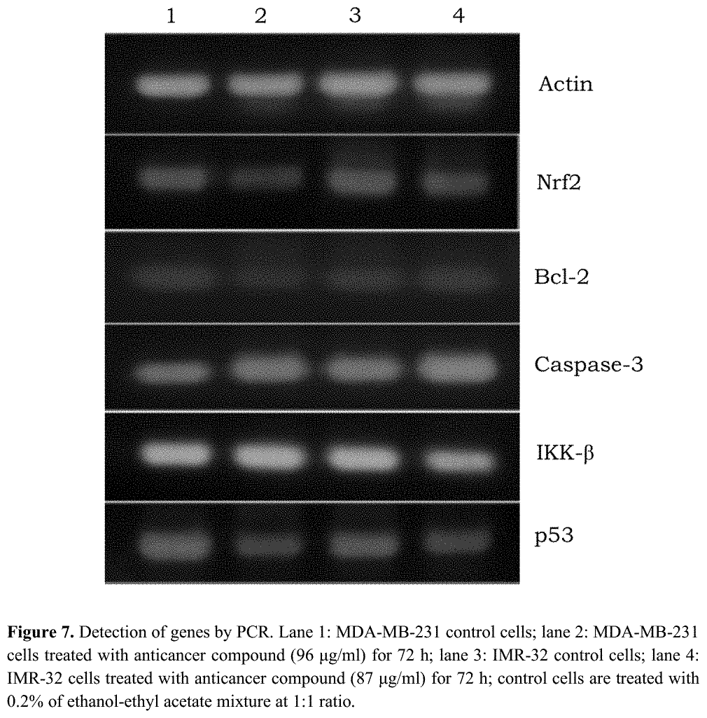

Genetic level changes associated with the treatment of anticancer compound in MDA-MB-231 and IMR-32 cells were studied by isolating the RNA, reverse transcribing the RNA to cDNA, amplifying the genes using specific primers. The amplified gene product when run on agarose gel was seen as shown in FIG. 7 under UV light. The resulting bands were analyzed using image J and the expression of each gene was represented as relative gene expression using the β-actin gene as a control.

During any stress condition, the Nrf2 translocates into the nucleus and binds to the antioxidant response element (ARE) present on the promoter region of the cytoprotective anti-apoptotic gene, Bcl-2. This lead to reduced apoptosis and enhanced cell survival (Kaspar et al., 2009).

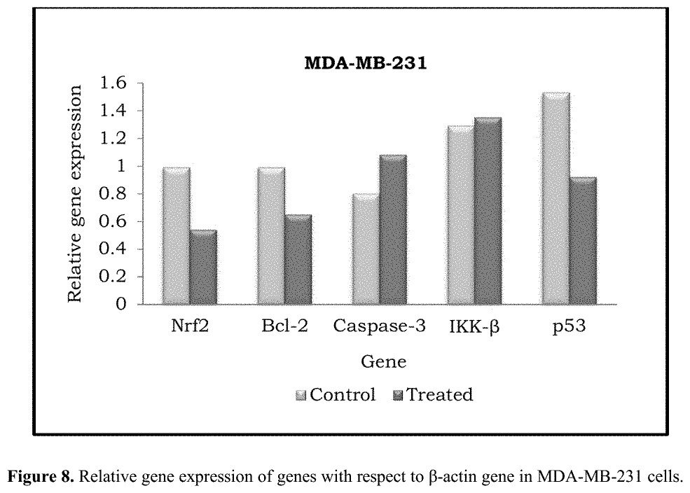

Treatment of MDA-MB-231 cells with anticancer compound for 72 h induced downregulation of transcription factor, Nrf2 when compared to control as shown in FIG. 8. This further resulted in reduced expression levels of Bcl-2 gene and upregulation of the pro-apoptotic gene, caspase-3 expression. This led to an increased apoptosis rate in cells treated with the anticancer compound. Similar type of cascading gene expressions was reported when cell underwent apoptosis in mouse hepatoma cells (Hepa-1), human hepatoblastoma cells (HepG2) (Niture & Jaiswal, 2012) and also in human glioblastoma cell (U251) (Pan et al., 2013). The expression level of IKK-β gene is almost similar in treated and control cells.

In apoptosis, caspase-3 is activated by both intrinsic and extrinsic pathways. During apoptosis, the IKK-β is proteolytically cleaved by the activated caspase-3 resulting in the decrease in the expression of IKK-β (Tang et al., 2001).

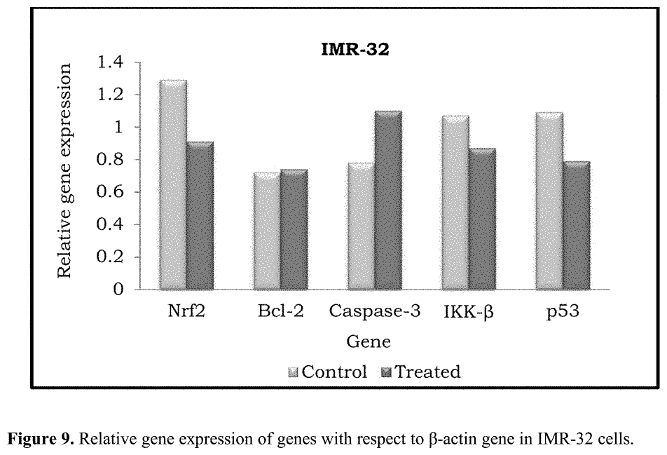

Treatment of IMR-32 cells with anticancer compound for 72 h resulted in upregulation of caspase-3 accompanied by downregulation of IKK-β as shown in FIG. 9, indicating that cell death occurred due to apoptosis. Also, there is a reduced expression level of Nrf2 but no change in the expression levels of Bcl-2 gene was observed in treated and control cells, indicating that the apoptotic pathway is independent of Bcl-2.

Further, the expression level of p53 gene in both MDA-MB-231 and IMR-32 treated cells was reduced when compared to control cells indicating that p53 gene is mutated in which case mutant p53 not only loses its pro-apoptotic activity but gains anti-apoptotic activity. (Rivlin et al., 2011).

### Example 3: Evaluation of Structure of Anticancer Compound According to Invention

### Liquid Chromatography-Mass Spectroscopy (LC-MS) Analysis of the Anticancer Compound:

LC-MS analysis of anticancer compound was carried out in Dionex Ultimate 3000 (Thermo) micro-LC equipped with C18, 150 mm×4.6 mm, 5 μm reverse phase column and ESI-QUAD-QTOF high-resolution mass spectrometer (Bruker impact HD). The solvents used were 0.1% formic acid in water (A) and 0.1% formic acid in acetonitrile (B). Table 7 shows the mobile phase parameters. LCMS analysis was done at LCMS facility, Indian Institute of Science, Bangalore.

TABLE 7
LC-MS mobile phase parameter

| Time (min) | 0 | 20 | 25 | 25.1 | 26 | 34 | 35 |
|---|---|---|---|---|---|---|---|
| Flow (ml/min) | 0.5 | 0.5 | 0.5 | 0.5 | 1.2 | 1.2 | 0.5 |
| Solvent A (%) | 90 | 5 | 5 | 98 | 98 | 98 | 95 |
| Solvent B (%) | 10 | 95 | 95 | 2 | 2 | 2 | 5 |

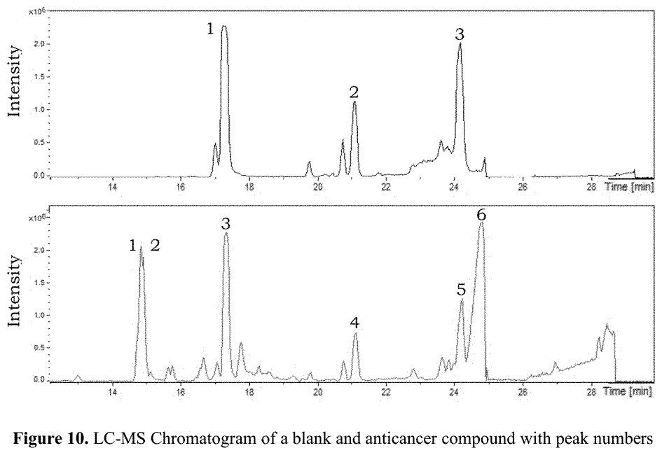

The Liquid Chromatography-Mass Spectroscopy (LC-MS) analysis data of the anticancer compound along with the blank is shown in FIG. 10. The major peaks in both blank and anticancer compound along with their Mass (m/z value), elution time and intensity of peak are shown in Tables 8 and 9 respectively.

TABLE 8
Mass (m/z value), elution time and intensity of peaks in a blank.

| | Elution time | m/z value | |
|---|---|---|---|
| Peak No. | (min) | (dalton) | Intensity |
| 1 | 17.4 | 186.1879 | 2295420 |
| 2 | 21.2 | 228.2348 | 1153558 |
| 3 | 24.1 | 270.2816 | 2028476 |

TABLE 9
Mass (m/z value), elution time and intensity
of peaks in the anticancer compound

| | Elution time | m/z value | |
|---|---|---|---|
| Peak No. | (min) | (dalton) | Intensity |
| 1 | 14.9 | 334.2956 | 2061026 |
| 2 | 15.0 | 290.2695 | 1900496 |
| 3 | 17.4 | 186.1863 | 2287126 |
| 4 | 21.2 | 228.2330 | 736378 |
| 5 | 24.3 | 270.2795 | 1271154 |
| 6 | 24.5 | 338.3421 | 2431266 |

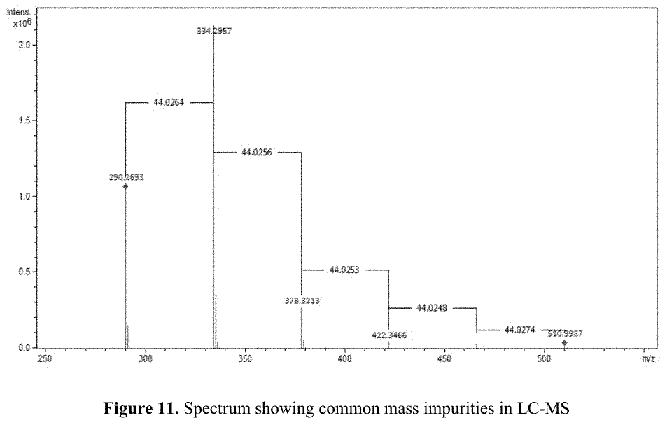

When we compare the blank and the anticancer compound peaks 1, 2 and 3 in blank are same as peaks 3, 4 and 5 in anticancer compound respectively. Therefore peaks 1, 2 and 6 are the three major peaks in the anticancer compound with m/z value of 334.2956 Da, 290.2695 Da and 338.34 Da respectively. Among these three, peaks 1 and 2, which are prominent in sample 16 are polymer impurities. To support this, a spectrum that shows the common mass impurities in LC-MS is shown in FIG. 11. Masses of peaks 1 and 2 are present among these. It also shows other masses which together makes a cluster of masses all separated exactly by 44.02 Da. These impurities primarily come from the plasticware used during sample preparation.

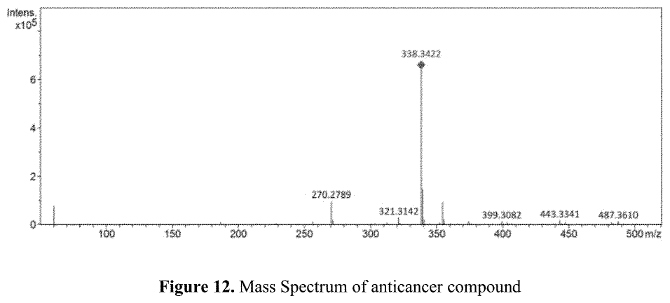

Finally, in the anticancer compound, peak 6 corresponds the actual molecular peak with a mass of 338.34 Da, which is also evident from FIG. 10. The mass spectrum of the anticancer compound was shown in FIG. 12. When the mass library was searched for the compounds with a mass of 338 Da, it is in good agreement with the empirical formula C₂₄H₃₅N.

### Nuclear Magnetic Resonance (NMR) Analysis the Anticancer Compound:

NMR spectrum gives information about the structure of the compound. The NMR studies were carried out in Centre for Cellular and Molecular Platforms (C-CAMP), Bangalore. The purified sample was dissolved in deuterochloroform (CDCl₃) and placed in between two strong magnets in Bruker Avance III HD 800 MHz instrument. The different chemical shifts of the proton and carbon according to their molecular environments within the molecule were determined with tetramethylsilane (TMS) as an internal standard. In order to determine the structure of the anticancer compound, ¹H NMR, ¹³C NMR and 2D-NMR studies were carried out.

### Proton NMR (¹H NMR) Analysis:

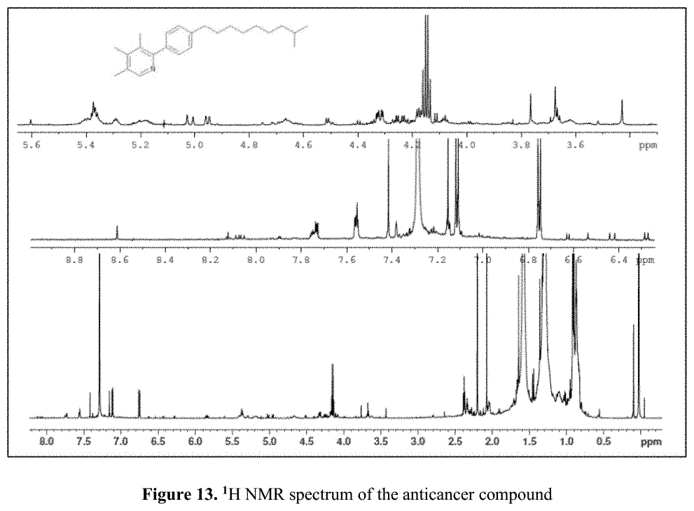

The compound shows a singlet at δ 7.45 for Pyridine —H (3) and doublet appeared at δ 7.21-7.20 (11, 15) and δ 6.75-6.74 (12, 14) due to aromatic protons. The methyl protons (8) appeared at δ 2.43-2.41 as a singlet, the remaining methyl protons (9, 10) showed singlet and benzylic protons (16) appeared as triplet at δ 2.19-2.21 ppm. The —CH₂ and —CH (17, 23) protons exhibited as multiplet at δ 1.49-1.48 ppm and multiplet appeared due to the presence of —CH₂ protons (18, 19, 20, 21, 22). A multiplet was also observed at δ 0.87 ppm for the presence of two methyl protons (24, 25). ¹H NMR spectrum of the anticancer compound is shown in FIGS. 13 and ¹H chemical shifts in the anticancer compound according to invention is shown in Table 10.

TABLE 10
¹H chemical shifts in the anticancer compound

| Proton No. | δ Values |
|---|---|
| 3 | 7.45 (s, 1H, Py-H) |
| 11, 15 | 7.21-7.20 (d, J = 3.6 Hz, 2H, Ar—H) |
| 12, 14 | 6.75-6.74d, J = 3.6 Hz, 2H, Ar—H) |
| 8 | 2.43-2.41 (m, 3H) |
| 9, 10, 16 | 2.19-2.21 (s, 8H, 2x-CH₃—CH₂) |
| 17, 23 | 1.49-1.48 (m, 3H, —CH₂, —CH) |
| 18, 19, 20, 21, 22 | 1.41-1.22 (m, 10H, 5x-CH₂) |
| 24, 25 | 0.87 (m, 6H 2x-CH₃) |

¹H NMR (CDCl₃, 800 MHz): δ 7.45 (s, 1H), 7.21-7.20 (d, J=3.6 Hz, 2H), 6.75-6.74 (d, J=3.6 Hz, 2H), 2.43-2.41 (m, 3H), 2.19 (s, 8H), 1.49-1.48 (m, 3H), 1.41-1.22 (m, 10H), 0.87 (m, 6H).

### Carbon NMR (¹³C NMR) Analysis:

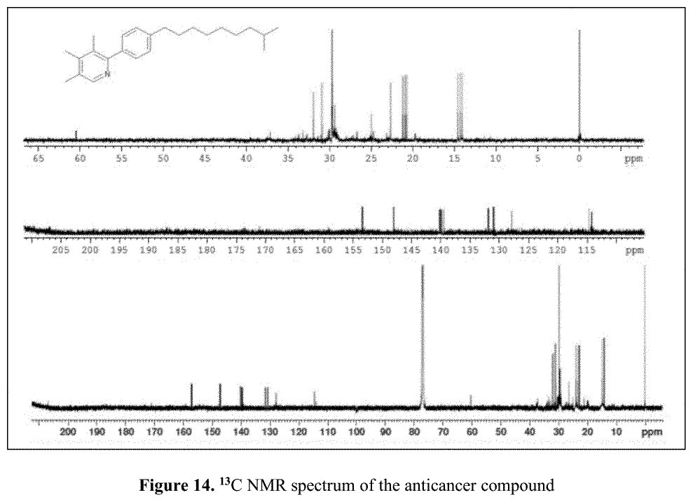

The compound shows a signal at δ 14.1, δ 14.2 and δ 14.4 for three —CH₃ groups (8, 9 10) present on the pyridine ring. Two singlets appeared at δ 22.5 and δ 22.6 due to two methyl carbons (24, 23) which are present on aliphatic chain and the remaining —CH₂ carbons (21, 18, 19, 20, 17, 16, 22) show up as signals in the range of δ 22.7-32.3 ppm. ¹³C NMR spectrum of the anticancer compound is shown in FIG. 14 and ¹³C chemical shifts in the anticancer compound is shown in Table 11.

TABLE 11
¹³C chemical shifts in the anticancer compound

| | δ | | |
|---|---|---|---|
| Carbon No. | Values | Carbon No. | δ Values |
| 8 | 14.1 | 22 | 32.3 |
| 10 | 14.2 | 11 | 114.9 |
| 9 | 14.4 | 2, 16 | 115.8 |
| 24, 25 | 22.5 | 12, 14 | 130.1 |
| 23 | 22.6 | 6 | 130.2 |
| 21 | 22.7 | 7 | 132.6 |
| 18, 19, 20 | 24.9 | 13 | 139.8 |
| 17 | 9.6 | 1 | 140.2 |
| 16 | 1.1 | 3 | 147.6 |

¹³ CNMR (CDCl₃): 14.1, 14.2, 14.4, 22.5, 22.6, 22.7, 24.9, 29.6, 31.1, 32.3, 114.9, 115.8, 130.1, 130.2, 132.6, 139.8, 140.2, 147.6, 157.8; MS: m/z 337.54 (M + 1).

### 2D-NMR Analysis

### Heteronuclear Multiple Bond Correlation (HMBC) Analysis:

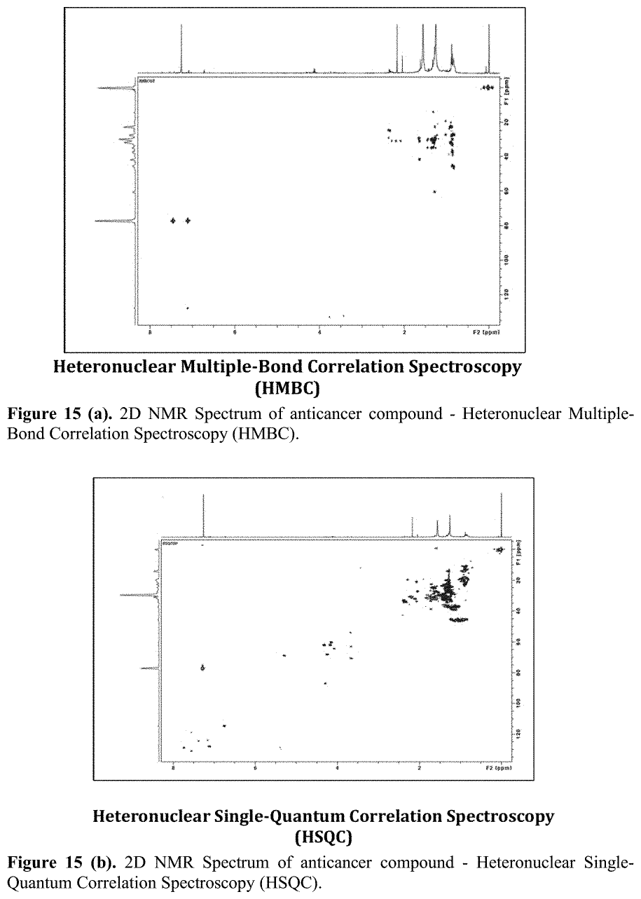

Beginning with the methyl groups, both CH₃ (24) and CH₃ (25) share a vicinal COSY correlation to 23H (identified by HSQC as a methine). This suggests a isopropyl fragment, confirmed by the mutual HMBC and long-range COSY correlations between 24H and 25H. 23H further has a vicinal COSY coupling to 22H (HSQC identifies this as a methine), suggesting fragment —CH—(CH₃)₂. 22H has additional COSY correlations to 21H and 20H. The connectivity of fragment —C₁₉H—C₁₈H₂—C₁₇H—C₁₆H is supported by HMBC correlations 12H-17H (130.1 ppm-29.6 ppm) and 15H-16H (114.9 ppm-31.1 ppm). The pyridine-3H is desheilded because of the Nitrogen atom in the pyridine ring that appeared at 7.45 ppm as singlet. As three bond coupling between 13C and H is stronger than the two bonds coupling in the aromatic system, the strong HMBC contour between the hydrogen at 7.21-7.16 ppm and the carbon at 157.8 ppm helps to identify the ipso carbon of the C-7 phenyl ring. Similarly, the strong HMBC contour between the hydrogen at 7.21-7.16 ppm and the carbon at 139.8 ppm reveals the phenyl group. The FIG. 15 (a) shows 2D NMR Spectrum of anticancer compound—Heteronuclear Multiple-Bond Correlation Spectroscopy (HMBC).

### Heteronuclear Single Quantum Coherence (HSQC) Analysis:

This data indicates clearly which proton is attached to which carbon. A carbon at 22.6 ppm is directly bonded to proton at 1.48 ppm. The phase of this correlation and the values of chemical shifts indicate that this is a methine carbon (—CH₂₃). For 18C, 19C, 20C, 21C and 22C, the signals at 22.7-32.3 ppm of the —CH₂— groups are directly bonded to the proton at 1.41-1.22 ppm. This creates a pattern of -18CH₂-19CH₂-20CH₂, 21CH₂-22CH₂— in aliphatic chain link. In a similar fashion the assignments can be completed through the rest of the ring. A signal at 14.1 ppm shall be assigned to the methyl carbon (8C) bonded to protons at 2.43 ppm as singlet. Another signals at 14.4 ppm for 9C shows the directly bonded aromatic carbon (1C). This 9C is then correlated to 2.46 ppm that are previously assigned to the carbon (3C) at 147.6 ppm. Thus it is completely assigned to all of 1H and 13C chemical shifts of the atoms within the pyridine ring of the compound. FIG. 15 (b) shows 2D NMR Spectrum of anticancer compound—Heteronuclear Single-Quantum Correlation Spectroscopy (HSQC).

The above furnished spectral data along with 2D-NMR (HMBC, HSQC) studies have provided great evidence supporting the positions of the groups.

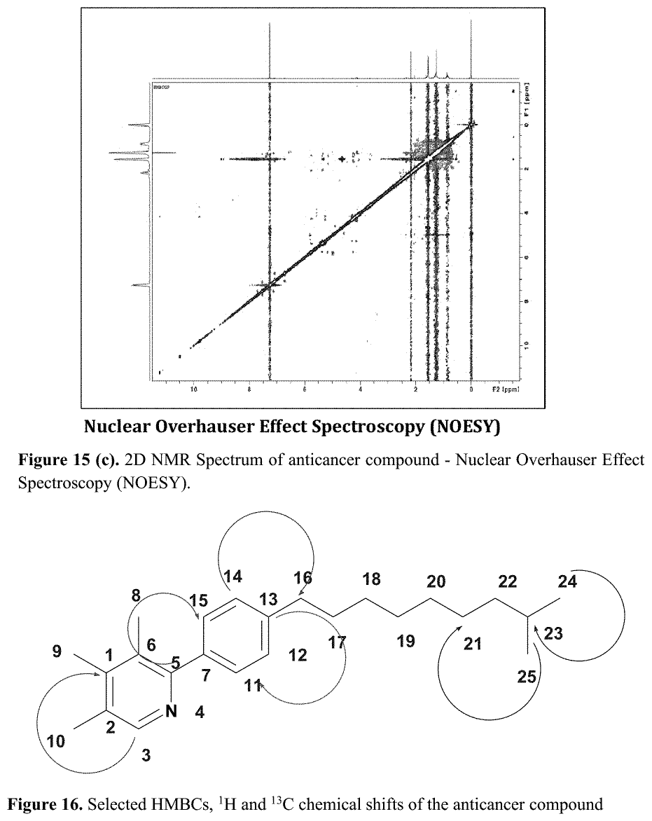

Further, 2D NMR Spectrum of anticancer compound—Nuclear Overhauser Effect Spectroscopy (NOESY) was also carried and shown in FIG. 15(c). Selected HMBCs, ¹H and ¹³C chemical shifts of the anticancer compound have been shown in FIG. 16.

### Structure of the Anticancer Compound:

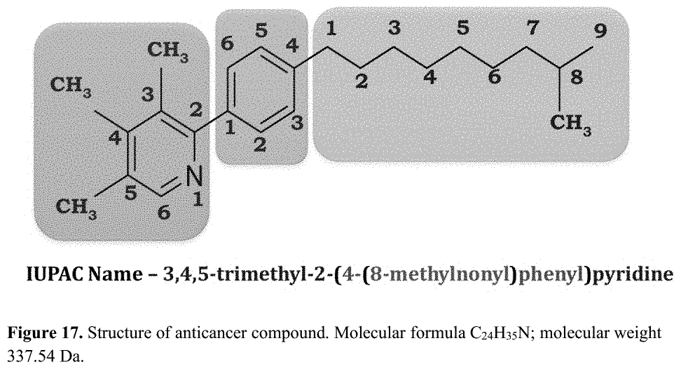

Based on the data of LCMS, ¹H NMR, ¹³C NMR, 2D-NMR and their elemental analysis, the name of the compound is found to be 3, 4, 5-trimethyl-2-(4-(8-methylnonyl)phenyl)pyridine with the structure shown in FIG. 17. The Molecular Formula for the compound of invention is C₂₄H₃₅N and Molecular weight is 337.54 Da.

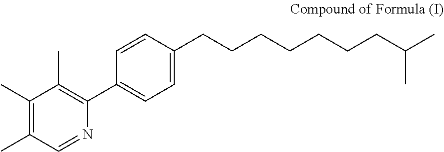 [SMILES: Cc1cnc(-c2ccc(CCCCCCCC(C)C)cc2)c(C)c1C]

Analysis calculated for C₂₄H₃₅N—C, 85.40; H, 10.45; N, 4.15; Found: C, 85.36; H, 10.41; N, 4.13.

While the present disclosure has been illustrated and described with respect to a particular embodiment thereof, it should be appreciated by those of ordinary skill in the art that various modifications to this disclosure may be made without departing from the spirit and scope of the present disclosure.

What is claimed is:

1. A compound of Formula (I) 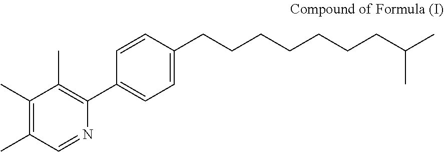 [SMILES: Cc1cnc(-c2ccc(CCCCCCCC(C)C)cc2)c(C)c1C]
or a pharmaceutically acceptable salt thereof.

2. The compound of Formula (I) as claimed in claim 1, wherein the compound is 3, 4, 5-trimethyl-2-(4-(8-methylnonyl)phenyl)pyridine.

3. The compound of Formula (I) as claimed in claim 1, wherein the compound is obtained by isolation from Sesuvium portulacastrum.

4. The compound of Formula (I) as claimed in claim 1, wherein the compound is an anticancer agent.

5. The compound of Formula (I) as claimed in claim 1, wherein the compound is effective against human triple negative breast cancer MDA-MB-231 cells, estrogen positive breast cancer MCF-7 cells, human neuroblastoma IMR-32 cells and human colon cancer HCT-116 cells.

6. A process for the isolation of compound of Formula (I) from Sesuvium portulacastrum, 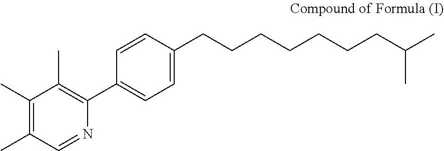 [SMILES: Cc1cnc(-c2ccc(CCCCCCCC(C)C)cc2)c(C)c1C]
said process comprising the steps of:
i. obtaining powder of dried whole plant of Sesuvium portulacastrum,
ii. extracting the powdered Sesuvium portulacastrum with one or more solvent,
iii. concentrating the extract obtained in step (ii) to obtain crude extract,
iv. optionally, fractionating the crude extract by column chromatography,
v. separating and purifying the crude extract obtained in step (iii) or the fractions obtained in step (iv) by preparative TLC and
vi. isolating the compound of Formula (I).

7. The process as claimed in claim 6, wherein the solvent used in step (ii) is selected from the group comprising of methanol, ethanol, acetone, hexane, ethyl acetate, dichloromethane, diethyl ether or mixture thereof.

8. The process as claimed in claim 6, wherein the column chromatography is carried by using the solvent system comprising hexane, ethyl acetate and dichloromethane in 50:5:45 ratio.

9. The process as claimed in claim 6, wherein the preparative TLC is carried by using the solvent system comprising hexane, ethyl acetate, dichloromethane and acetone in 49:5:44:2 ratio.

10. A pharmaceutical composition comprising a pharmaceutically effective amount of the compound of Formula (I) and one or more pharmaceutical excipient.

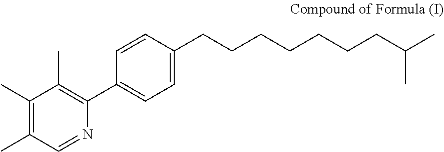 [SMILES: Cc1cnc(-c2ccc(CCCCCCCC(C)C)cc2)c(C)c1C]

11. Use of compound of Formula (I) or a pharmaceutically acceptable salt thereof for the treatment of cancer

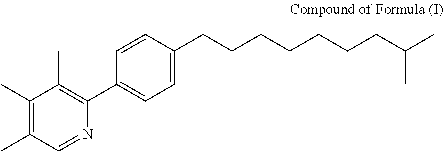 [SMILES: Cc1cnc(-c2ccc(CCCCCCCC(C)C)cc2)c(C)c1C]

12. A method for treating cancer in a subject comprising administering an effective amount of compound of Formula (I) or a pharmaceutically acceptable salt thereof. 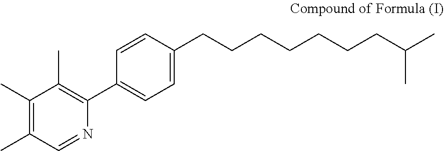 [SMILES: Cc1cnc(-c2ccc(CCCCCCCC(C)C)cc2)c(C)c1C]
